(19) United States
(12) Patent Application Publication
(10) Pub. No.: US 2024/0173460 A1
(43) Pub. Date: May 30, 2024

(54) STORAGE SOLUTIONS FOR MAINTAINING OF AXONAL VIABILITY

(71) Applicants: David M. JACKSON, Lago Vista, TX (US); Board of Regents, The University of Texas System, Austin, TX (US)

(72) Inventors: George D. BITTNER, Austin, TX (US); David M. JACKSON, Lago Vista, TX (US)

(73) Assignee: Board of Regents, The University of Texas System, Austin, TX (US)

(21) Appl. No.: 18/514,277

(22) Filed: Nov. 20, 2023

Related U.S. Application Data
(60) Provisional application No. 63/384,710, filed on Nov. 22, 2022.

(51) Int. Cl. A61L 27/50; A61L 27/02; A61L 27/04; A61L 27/38

(52) U.S. Cl. CPC A61L 27/50; A61L 27/025; A61L 27/047; A61L 27/383; A61L 2300/802; A61L 2430/32

(57) ABSTRACT

The disclosure relates to defined solutions for organ (heart, liver, kidney, etc,) vascular composite allografts or autografts (VCA: hand, limb, face, etc.) or tissue (peripheral nerves, spinal cord, skin) storage that preserve the electrophysiological functions of axons contained therein. The disclosure also concerns the use of isolated peripheral nerve segments and solid organs preserved in the defined solutions in autograft or allograft transplantation procedures, as well as the use of isolated peripheral nerve segments and solid organs preserved in the defined solutions in autograft/allograft transplantation procedures in combination with the peripheral nerve repair techniques of neurorrhaphy and axonal fusion primarily using polyethylene glycol known as PEG-fusion.

### PRIORITY CLAIM

This application claims benefit of priority to U.S. Provisional Application Ser. No. 63/384,710, filed Nov. 22, 2022, the entire contents of which are hereby incorporated by reference.

### STATEMENT REGARDING FEDERALLY FUNDED RESEARCH

This invention was made with government support under Grant no. W81XWH-19-2-0054 awarded by Army Medical Research and Material Command, ARMY/MRMC. The government has certain rights in the invention.

### BACKGROUND

### I. Field

The present disclosure relates to the fields of medicine and neurobiology. More particularly, it addresses the need for improved compositions and methods for treating and storing of nerve grafts prior to use in axon repair applications.

### II. Related Art

Characteristics of most allograft vs nerve allograft transplants stored ex vivo. Current protocols for transplantation of donor, non-neuronal organs (e.g., livers, hearts, kidneys) stored ex vivo are designed to restore vascular connections that re-establish oxygenated blood flow (Petrenko et al., 2019). The clinical intention for some tissue grafts of non-neuronal origin (e.g., skin, fascia) also stored ex vivo is often to produce progressive revascularization via capillary ingrowth within days to weeks after transplantation to maintain donor cell viability (Ramsey et al., 2022). The clinical intention for other non-neuronal, acellular tissue allografts (e.g., bone, cartilage, tendon) stored ex vivo is to maintain long-term structural integrity after transplantation with no requirement for immediate revascularization (Sohn and Oh, 2019). Immediate restoration and continued maintenance of function is the usual measure of success for all these transplanted organs or tissues that most commonly have been stored ex vivo for 1-2 days in University of Wisconsin cold storage solution (UW solution) that maintains donor cell viability (Spiegel et al., 1999; Chen et al., 2019).

In contrast to the organs and tissues stored ex vivo described above, the current surgical intent for segments of peripheral nerve stored ex vivo as a vascularized or non-vascularized tissue transplant is to provide a scaffold that may help axonal regeneration by outgrowth from proximal cut ends of host axons connected to their soma (Allan, 2000; Houschyar et al., 2016; Bittner et al., 2018). Distal segments of peripheral nerve axons within these tissues are expected to be non-functional and undergo WD within 3-7 days. None of the above examples of procedures for organ or tissues stored ex vivo are expected upon transplantation to rapidly re-establish the normal functions of the peripheral nerve axons. Hence, storage solutions like UW have only been tested for their ability to preserve the structure, and not axonal function, of peripheral nerve grafts and how stored peripheral nerve allografts affect host immunological responses to the grafts (Hare et al., 1993; Ikeguchi et al., 2007). We have addressed the problem of how to assess and preserve axonal structure and function in anucleate segments of peripheral nerves in ex vivo storage solutions.

Importance of nerve allograft transplants. The ability to preserve anucleate segments of axons in peripheral nerves is important because PNIs often produce nerve gap (segmental-loss) defects. Segmental-loss PNIs are the most common form of neural dysfunction (Bittner et al., 2022). Patients and experimental laboratory animals with nerve gap defects sequentially experience (1) immediate complete loss of sensory and motor functions mediated by the denervated structures followed by (2) rapid WD of severed distal axonal segments within a few days after injury, and (3) slow (˜1 mm/d) natural regeneration by outgrowths that produce poor (if any) functional recovery after months to years, due in part to non-specific reinnervation and/or to muscle atrophy or deterioration of nerve target structures before reinnervation (Gaudet et al., 2011; Menorca et al., 2013; Scheib and Hoke, 2013; Kornfeld et al., 2019; Bittner et al., 2022). Current best clinical or experimental practices to repair segmental-loss PNIs employ (1) autografts obtained from other previously intact nerves, usually sensory in nature, (2) donor non-allogenic (decellularized/denatured) allografts, or (3) synthetic conduits (Mackinnon et al., 2001; Muheremu and Ao, 2015; Pan et al., 2020). All these natural or artificial conduits are micro-sutured to host epineurium and connective tissue at both ends of the gap to create a bridge across the gap to guide surviving proximal axons to distal nerve segments.

All these contemporary procedures to repair nerve gaps are problematic because they do not prevent WD of distal axonal segments and, therefore, solely rely on natural regeneration to produce slow and poor recovery of motor function and/or sensation. The lack of supporting cells in acellular nerve grafts and artificial conduits is believed to reduce their efficacy (Pan et al., 2020). Despite previous attempts using freshly harvested peripheral nerve transplants that have viable axons and other supporting cells, the repairs have not been successful; transplanted tissues were often rejected even with systemic immune suppression and major and/or minor histocompatibility complex matching (Mackinnon et al., 2001; Campbell, 2008). Alternatively, a recently-developed repair strategy using polyethylene glycol (PEG) fusion of peripheral nerves with viable axons (PNVAs) to repair segmental-loss PNIs has produced excellent recovery of function and voluntary behaviors within weeks (Riley et al., 2015; Bittner et al., 2016a). Furthermore, PEG-fused PNVAs showed minimal immune response despite neither histocompatibility complex matching nor immune suppression (Mikesh et al., 2018a).

### SUMMARY

Thus, in accordance with the present disclosure, there is provided a nerve storage solution comprising calcium-free solution comprising sodium chloride, sodium acetate anhydrous, sodium gluconate, potassium chloride, and magnesium chloride and exhibiting no more than about 250-255 mOsm per liter (measured osmosality). The sodium chloride may be present at about 526 mg per 100 ml, and/or the sodium acetate may be present at about 222 mg per 100 ml, and/or the sodium gluconate may be present at about 502 mg per 100 ml, and/or the potassium chloride may be present at about 37 mg per 100 ml, and/or the magnesium chloride hexahydrate may be present at about 30 mg per 100 ml. In particular embodiments the sodium chloride is present at about 526 mg per 100 ml, sodium acetate is present at about 222 mg per 100 ml, sodium gluconate is present at about 502 mg per 100 ml, potassium chloride is present at about 37 mg per 100 ml, and magnesium chloride hexahydrate is present at about 30 mg per 100 ml. The nerve storage solution may be further defined as (a) comprising hydrochloric acid and/or sodium hydroxide; (b) having a pH range 6.5 to 7.6; (c) having electrolytes per 1000 mL (not including pH adjustment) of sodium about 140 mEq, potassium about 5 mEq, magnesium about 3 mEq, chloride about 98 mEq, acetate about 27 mEq, and gluconate about 23 mEq; and (d) (i) comprising no bacteriostat, antimicrobial agent or added buffer (except for pH adjustment) or (ii) comprising a bacteriostat or antimicrobial agent, with or without added buffer, such as one or more of penicillin, streptomycin, amphotericin B, kanamycin, gentamicin sulfate, tylosin, chloramphenicol, ampicillin, neomycin, benzylpenicillin, hygromycin B, carbenicillin, crythromycin, cefotaxime, paromomycin and fosmidomycin, or combinations thereof, including penicillin+streptomycin. The nerve storage solution may be hypotonic Normosol-R (dNR). The nerve storage solution may further comprise an axon or a tissue comprising an axon.

In another embodiment, there is provided a method of repairing an injury to a nerve site in a subject comprising (a) providing a nerve graft comprising an axon; (b) treating said nerve graft with a solution as defined herein; (c) placing the nerve graft into the injured nerve site. The injured nerve site may be in a peripheral nerve, and/or may be in an organ, such as heart, lung, kidney, liver, colon, intestine, esophagus, tongue, or stomach. The injured nerve site may be in an arm, a leg, a finger or a toe. The nerve graft may be derived from a sensory nerve, motor nerve, or a mixed nerve. The nerve graft may be an allograft, such as a vascularized composite allograft, isograft or an autograft. The subject may be a human subject or a non-human mammalian subject. The injury may be an acute injury or a chronic injury. The nerve graft may be treated with polyethylene glycol.

In still yet another embodiment, there is provided a method of treating or storing an axon or tissue containing an axon comprising contacting said axon or tissue containing an axon with a nerve storage solution comprising calcium-free solution comprising sodium chloride, sodium acetate anhydrous, sodium gluconate, potassium chloride, and magnesium chloride and exhibiting no more than about 250-255 mOsm per liter (measured osmosality). The sodium chloride may be present at about 526 mg per 100 ml, and/or the sodium acetate may be present at about 222 mg per 100 ml, and/or the sodium gluconate may be present at about 502 mg per 100 ml, and/or the potassium chloride may be present at about 37 mg per 100 ml, and/or the magnesium chloride hexahydrate may be present at about 30 mg per 100 ml. In particular embodiments the sodium chloride is present at about 526 mg per 100 ml, sodium acetate is present at about 222 mg per 100 ml, sodium gluconate is present at about 502 mg per 100 ml, potassium chloride is present at about 37 mg per 100 ml, and magnesium chloride hexahydrate is present at about 30 mg per 100 ml. 28.

[symbol]

The nerve storage solution may be further defined as (a) comprising hydrochloric acid and/or sodium hydroxide; (b) having a pH range 6.5 to 7.6; (c) having electrolytes per 1000 mL (not including pH adjustment) of sodium about 140 mEq, potassium about 5 mEq, magnesium about 3 mEq, chloride about 98 mEq, acetate about 27 mEq, and gluconate about 23 mEq; and (d) (i) comprising containing no bacteriostat, antimicrobial agent or added buffer (except for pH adjustment) or (ii) comprising a bacteriostat or antimicrobial agent, with or without added buffer. The nerve storage solution may be hypotonic Normosol-R (dNR). The treating or storing may occur for about 4-10 days at a temperature of about 4° C. to about 25° C. The treating or storing may occur for about 4 or 5 days at a temperature of about 25° C. The treating or storing may occur for about 9 or 10 days at a temperature of about 4°C. The method may further comprise testing the axon or tissue containing an axon for one or more of viability, axonal integrity, myelin integrity, tissue disintegration, and/or compound action potential. One or more of viability, axonal integrity, myelin integrity, tissue disintegration, and/or compound action potential, may be improved as compared to an untreated axon or tissue containing an axon.

Also provided is an axon or tissue comprising an axon treated with a nerve storage solution as defined herein.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” means 5%±the stated numerical value for a given attribute, feature or embodiment. Alternatively, “about” is used to indicate that a value includes the inherent variation of error for measurement of the attribute, feature or embodiment, the method being employed to determine the value, or the variation that exists among the sample.

It is contemplated that any embodiment discussed in this specification can be implemented with respect to any method or composition of the invention, and vice versa. Furthermore, compositions and kits of the invention can be used to achieve methods of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-F. Recording chamber and CAPs. (FIG. 1A) Recording chamber. SE: stimulating electrode. (−) and (+) indicate two ends of the recording electrode. (FIGS. 1B-F) CAP: compound action potential from PNVA in 4° C. dNR. SA: stimulus artifact.

FIGS. 2A-I. Nerve graft survival in various storage conditions. (FIG. 2A) 4° C. dNR from male (n=6) and female (n=32) SD rats. (FIG. 2B) 4° C. dNR from from wild-type SD rats (n=10) and Thy1-GFP SD rats (n=12). (FIG. 2C) 4° C. dNR from nerves operated and tested by two different surgeons (MA n=10, TZ n=22). (FIGS. 2D-I) plots the composite CAP conduction data (n=38) stored in 4° C. dNR against other storage conditions in different panels as follows: 25° C. dNR (FIG. 2D), 4° C. NR (FIG. 2E), 4° C. dNRc and 25° C. dNRc (FIG. 2F), 4° C. LR (FIG. 2G), 4° C. dNS, 4° C. NS and 25° C. NS (FIG. 2H), and 4° C. UW (FIG. 2I). *p<0.05, **p<p<0.01, ***p<0.001, ****p<0.0001; significance levels were the same using both Log-rank test and Gehan-Breslow-Wilcoxon test. ns, not significant. n numbers were indicated in colored font at the bottom left corner of each panel. Some nerve samples were used for other analyses on day 3, and decreased n numbers are indicated. Note that n (number of nerves stored in composite curve 4° C. dNR) is given only in FIG. 2D because that n is the same for the composite curve 4° C. dNR repeated in FIGS. 2D-I.

FIG. 3. Summary of % PNVA's that conducted CAPS at 0-10d storage times.

FIGS. 4A-E. Representative histological sections of PNVAs showing five axonal/myelin categories. (FIG. 4A) Well-organized myelin and axoplasm. (FIG. 4B) Abnormal Myelin. (FIG. 4C) Abnormal axoplasm. (FIG. 4D) Abnormal myelin and axoplasm. (FIG. 4E) Disintegrating myelin. White arrows point to well-organized axons. White asterisks point to slight myelin dissociation, possibly a cleft of Schmidt-Lanterman). Blue arrows point to retracted axoplasm. Red arrows point to myelin dissociation and intrusion. Orange arrows point to myelin disintegration. Scale bar=5 μm.

FIGS. 5A-F. Morphological analyses of axons in stored PNVAs. (FIGS. 5A-E) representative thick sections of PNVAs stored for 0, 1, 3, and 5 days in 4° C. dNR and 1 day in 4° C. NS. Scale bar=10 μm. (FIG. 5F) Organization of myelin and axoplasm according to the five-category classification system. Data are plotted as percent of axons (mean±s.e.m) under different morphological categories. Two-way ANOVA comparison F₍16,60)=23.52, p<0.0001. Two-way ANOVA followed by Tukey's multiple comparison's test (post-hoc results in Table 3). n=3-4 nerves (at least 150 axons per nerve) for each condition.

### DETAILED DESCRIPTION

The inventors, seeking an improved nerve/tissue graft preservation and storage solution, examined several commonly used organ/tissue storage solutions (University of Wisconsin Cold Storage Solution, Normosol-R, Normal Saline, and Lactated Ringers), including those with anti-microbial agents, for axonal viability in sciatic nerves ex vivo as assessed by maintaining (1) conduction of compound action potentials (CAPs) and (2) axonal and myelin morphology in a novel assay method. The ten different storage solution conditions for peripheral nerves with viable axons (PNVAs) differed in their solution composition, osmolarity (250-318 mOsm/liter), temperature (4° C. versus 25° C.), and presence of calcium. They observed that CAPS and axonal morphology in PNVAs can best be maintained for up to 9 days in calcium-free hypotonic diluted (250 mOsm/liter) Normosol-R (dNR) at 4° C. Surprisingly, CAPs and axonal morphology were maintained for only 1-2 days in UW and NS at 4° C., much less than maintained in 4° C. dNR (9 days) or even in dNR (5 days) at 25° C.

These observations are significant in that maintaining axonal viability in PNVAs is important because only viable donor axons in PNVAs can be fused (e.g., treated with polyethylene glycol or “PEG”) with viable proximal and distal ends of host axons to repair segmental-loss peripheral nerve injuries. PEG-fused PNVAs produce excellent recovery of sensory/motor functions and voluntary behaviors within weeks. Such PEG-fused PNVAs, unlike conventional donor transplants of sciatic nerves or other tissue/organ allografts, are immune-tolerated without tissue matching or immune suppression. Furthermore, the ability to maintain axonal viability in stored PNVAs would make possible the establishment of PNVA tissue banks to address the current shortage of transplantable nerve grafts and the use of stored, PEG-fused PNVAs to repair segmental loss PNIs.

These and other aspects of the disclosure are set out in detail below.

### I. Peripheral Nerve Injuries

Nerve injury is any injury to nervous tissue. Unlike in the central nervous system. neuroregeneration in the peripheral nervous system is possible. The processes that occur in peripheral regeneration can be divided into the following major events: Wallerian degeneration, axon regeneration/growth, and reinnervation of nervous tissue. The events that occur in peripheral regeneration occur with respect to the site of the nerve injury. The proximal stump refers to the end of the injured neuron that is still attached to the neuron cell body; it is the part that regenerates. The distal stump refers to the end of the injured neuron that is still attached to the end of the axon; it is the part of the neuron that will degenerate, but the stump remains capable of regenerating its axons.

Wallerian degeneration is a process that occurs before nerve regeneration and can be described as a cleaning or clearing process that essentially prepares the distal stump for reinnervation. Schwann cells are glial cells in the peripheral nervous system that support neurons by forming myelin that encases nerves. During Wallerian degeneration Schwann cells and macrophages interact to remove debris, specifically myelin and the damaged axon, from the distal injury site. Calcium has a role in the degeneration of the damaged axon. Bands of Büngner are formed when uninnervated Schwann cells proliferate and the remaining connective tissue basement membrane forms endoneurial tubes. Bands of Büngner are important for guiding the regrowing axon.

At the neuronal cell body, a process called chromatolysis occurs in which the nucleus migrates to the periphery of the cell body and the endoplasmic reticulum breaks up and disperses. Nerve damage causes the metabolic function of the cell to change from that of producing molecules for synaptic transmission to that of producing molecules for growth and repair. These factors include GAP-43, tubulin and actin. Chromatolysis is reversed when the cell is prepared for axon regeneration.

Axon regeneration is characterized by the formation of a growth cone, which has the ability to produce a protease that digests any material or debris that remains in its path of regeneration toward the distal site. The growth cone responds to molecules produced by Schwann cells such as laminin and fibronectin.

There is no single classification system that can describe all the many variations of nerve injuries. Classification of peripheral nerve injury assists in prognosis and determination of treatment strategy. Classification of nerve injury was described by Seddon in 1943 and by Sunderland in 1951. The lowest degree of nerve injury in which the nerve remains intact but signaling ability is damaged is called neurapraxia. The second degree in which the axon is damaged, but the surrounding connecting tissue remains intact, is called axonotmesis. The last degree in which both the axon and connective tissue are damaged is called neurotmesis.

The 1943 Seddon classification described three basic types of peripheral nerve injury that include Neurapraxia, which is a temporary interruption of conduction without loss of axonal continuity. In neurapraxia, there is a physiologic block of nerve conduction in the affected axons.

Other characteristics include sensory-motor problems distal to the site of injury, the endoneurium, perineurium, and the epineurium are intact, no Wallerian degeneration, conduction is intact in the distal segment and proximal segment, but no conduction occurs across the area of injury, recovery of nerve conduction deficit is full, and requires days to weeks, and EMG shows lack of fibrillation potentials (FP) and positive sharp waves.

Axonotmesis is the next class and involves loss of the relative continuity of the axon and its covering of myelin, but preservation of the connective tissue framework of the nerve (the encapsulating tissue, the epineurium and perineurium, are preserved). Other characteristics include Wallerian degeneration distal to the site of injury, sensory and motor deficits distal to the site of lesion, no nerve conduction distal to the site of injury (3 to 4 days post-injury), EMG shows fibrillation potentials (FP), and positive sharp waves (2 to 3 weeks post-injury), and axonal regeneration occur and recovery is possible without surgical treatment. Sometimes surgical intervention is required, due to scar tissue formation.

The third category is Neurotmesis, which is characterized by a total severance or disruption of the entire nerve fiber. Neurotmesis may be partial or complete. Other characteristics include Wallerian degeneration distal to the site of injury, a connective tissue lesion that may be partial or complete, severe sensory-motor problems and autonomic function defect, no nerve conduction distal to the site of injury (3 to 4 days after lesion), EMG and NCV findings are as axonotmesis, and because of lack of nerve, surgical intervention is necessary.

The 1951 Sunderland classification system expanded Seddon's classification to five degrees of peripheral nerve injury: first degree (same as Seddon's Neurapraxia), second degree (same as Seddon's Axonotmesis), third degree (included within Seddon's Neurotmesis) is a nerve fiber interruption. In third-degree injury, there is a lesion of the endoneurium, but the epineurium and perineurium remain intact. Recovery from a third-degree injury is possible, but surgical intervention may be required. Fourth degree (fourth degree is included within Seddon's Neurotmesis) has only the epineurium remaining intact. In this case, surgical repair is required. Fifth (included within Seddon's Neurotmesis) shows a complete transection of the nerve. Recovery is not possible without appropriate surgical treatment.

Surgery can be performed in case a nerve has become cut or otherwise divided. Recovery of a nerve after surgical repair depends mainly on the age of patients. Younger the patients, better the prognosis, because of better healing capacity of young tissues. Young children can recover almost normal nerve function. In contrast, a patient over 60 years old with a cut nerve in the hand would expect to recover only protective sensory function, that is, the ability to distinguish hot/cold or sharp/dull; recovery of motor function would be likely incomplete. Many other factors also affect nerve recovery. The use of autologous nerve grafting procedures that involve redirection of regenerative donor nerve fibers into the graft conduit has been successful in restoring target muscle function. Localized delivery of soluble neurotrophic factors may help promote the rate of axon regeneration observed within these graft conduits.

In terms of non-surgical treatments, electrical stimulation can promote peripheral nerve regeneration. The positive effect of electrical stimulation on nerve regeneration is due to its molecular influence on the damaged neuron and Schwann cells. Electrical stimulation can directly accelerate the expression of cyclic adenosine monophosphate (cAMP) both in neurons and Schwann cells. cAMP is a molecule that stimulates multiple signaling pathways that aid nerve regeneration by enhancing the expression of several neurotrophic factors. Electrical stimulation also results in the influx of calcium ions, which further triggers multiple regeneration pathways. The frequency of stimulation is an important factor in the success of both quality and quantity of axon regeneration as well as growth of the surrounding myelin and blood vessels that support the axon. Histological analysis and measurement of regeneration showed that low frequency stimulation had a more successful outcome than high frequency stimulation on regeneration of damaged sciatic nerves. Other studies have used both oscillating current (AC) and non-oscillating direct current (DC) stimulation to regenerate mammalian peripheral nerves. Mammalian neurons preferentially orient and grow towards the cathode in DC electric fields.

### II. Normosol-R® and Additives

### A. Normosol-R®

According to Drugs.com, Normosol®-R pH 7.4 is a sterile, nonpyrogenic, isotonic solution of balanced electrolytes in water for injection. It contains sodium chloride, sodium acetate anhydrous, sodium gluconate, potassium chloride, and magnesium chloride. The solution is typically administered by intravenous infusion for parenteral replacement of acute losses of extracellular fluid.

Each 100 mL of Normosol-R® pH 7.4 contains sodium chloride, 526 mg; sodium acetate, 222 mg; sodium gluconate, 502 mg; potassium chloride, 37 mg; magnesium chloride, hexahydrate 30 mg. May contain hydrochloric acid and/or sodium hydroxide for pH adjustment. pH range 6.5 to 7.6; 295 mOsmol/liter (calc.). Electrolytes per 1000 mL (not including pH adjustment): sodium 140 mEq; potassium 5 mEq; magnesium 3 mEq; chloride 98 mEq; acetate 27 mEq; gluconate 23 mEq. The solution contains no bacteriostat, antimicrobial agent or added buffer (except for pH adjustment).

Sodium Chloride, USP is chemically designated NaCl, a white crystalline powder freely soluble in water. Potassium Chloride, USP is chemically designated KCl, a white granular powder freely soluble in water. Magnesium Chloride, USP is chemically designated magnesium chloride, hexahydrate (MgCl₂·6H₂O) deliquescent crystals very soluble in water. Sodium Acetate, USP is chemically designated sodium acetate, anhydrous (C₂H₃NaO₂), a hygroscopic powder soluble in water. It has the following structural formula:

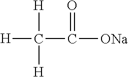

Sodium gluconate is chemically designated C₆H₁₁NaO₇, the normal sodium salt of gluconic acid soluble in water. It has the following structural formula:

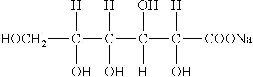

Water for Injection, USP is chemically designated H₂O.

### B. Antimicrobials

The solutions of the present disclosure may also advantageously include substances that will inhibit microbial growth. A wide variety of different agents are available for this purpose including penicillin, streptomycin, amphotericin B, kanamycin, gentamicin sulfate, tylosin, chloramphenicol, ampicillin, neomycin, benzylpenicillin, hygromycin B, carbenicillin, erythromycin, cefotaxime, paromomycin and fosmidomycin, or combinations thereof. The combination of penicillin and streptomycin is specifically contemplated.

### III. Nerve Grafts and Nerve Graft Fusions

### A. Peripheral Nerve Grafts

The nerve grafts of the present disclosure may be peripheral nerve allografts, isografts (grafts taken from an identical genotype but not the same organism, such as between identical twins or laboratory animal strains), autografts or xenografts. Nerve allotransplantation is the transplantation of a nerve to a recipient from a donor of the same species. For example, nerve tissue is transplanted from one person to another. Allotransplantation is a commonly used type of transplantation of which nerve repair is one specific aspect. In contrast, a nerve autograft is a nerve tissue removed from an individual for use in another site in the same individual. Autografts are transplants involving tissue from the same subject as both donor and recipient. Xenografts are transplants between donor and recipients of distinct species.

A nerve allograft is used for the reconstruction of nerve discontinuities in order to support the axonal regeneration across a nerve gap caused by any injury. Nerve tissue may be processed to remove cellular and non-cellular factors such as cells, fat, blood, axonal debris and chondroitin sulfate proteoglycans while preserving the three-dimensional scaffold and basal lamina tubular structure of the nerve. Such nerve allografts only contain extracellular matrix (ECM), which is sterile and decellularized.

There are three types of peripheral nerves: sensory nerves, which carry sensory information from peripheral organs (e.g., skin) to the central nervous system (responsible for sensation and proprioception); motor nerves—carry information from the central nervous system to peripheral organs (e.g., muscles) (nerve signal activity modulates muscle contraction, thereby enabling movement); mixed nerves containing both sensory and motor fibers.

In a trauma or surgical resection, a nerve can be damaged, which is called a nerve defect. This defect needs to be repaired in order to regain full or partial sensory and motor function. For example, peripheral nerve injury is a major clinical problem and can result in partial or complete short- or long-term loss of sensation and function as well as neuropathic pain, which is pain arising as a direct consequence of a lesion or disease affecting the somatosensory system. Damaged nerve fibers continuously excite electric pulses, inducing pain or abnormal sensation dysesthesia. It has been shown that in allograft surgeries, post-operative neuropathic pain was present in some patients, but only if they suffered from this condition pre-operatively. Patients without neuropathic pain before their surgery did not complain about neuropathic pain afterwards. Hence, allograft treatment does not seem to be a risk factor for this specific problem.

### B. Nerve Transplantation

There are several kinds of transplantation techniques. As discussed above, nerve auto-transplantation is transplantation within the same person. However, when there is a large nerve defect, there may be an insufficient number of nerves available for transplantation. The nervus suralis, a nerve from the lower leg, is often used. Consequently, the patient will miss the specific nerve used as an autograft. Therefore, a person's own nerves cannot be used for an unlimited number of nerve injury repairs. As such, allografts—nerve material from a genetically distinct subject of the same species—have grown in use over the past several decades. This both avoids additional injury to the subject and permits multiple surgical interventions on the same subject. Xenografts may also be used.

The transplant surgery generally involves the following steps. First, the surgeon prepares the broken nerve. This means the surgeon must examine the local tissue and resect scar tissue if needed. The proximal and distal segments of the injured nerves should be debrided to healthy tissue by visual and tactile signs. After that, the surgeon measures the distance between both nerve ends as well as the diameter of the damaged nerve. The surgeon then chooses between a nerve allograft or an autograft, but the procedure is essentially the same. In its most simple sense, this means sutures that connect the graft with the damaged nerve are placed in the epineurium. The present disclosure envisions two approaches—bridging transplants and spanning transplants. All of the important anatomical structures of the nerve are kept intact.

### C. Selecting and Preparing Nerve Graft Material

There are several factors that help a surgeon decide whether a nerve autograft or an allograft should be chosen. While the use of nerve autografts has some advantages, such as the lack of immune response by the subject, there are some drawbacks. One is that the surgeon always creates a defect in the donor from where the nerve is taken. Another disadvantage is that when the defect is large, the amount of available autograft material may be insufficient.

Nerve allografts bring a possible solution for some of these problems. Therefore, allografts can be used more often in the same patient than autografts. Studies suggest that nerve allografts work just as well as nerve autografts and are therefore a good alternative to the classic nerve autograft. One adverse effect of nerve allotransplantation is the immunogenic response. Tissue from another human being is used to restore the defect, which can induce an immunogenic response. An immune response against an allograft or xenograft is called transplant rejection. To prevent this rejection, one typically employs immunosuppressive techniques to the graft, before it is transplanted into the receiver. Allografts can be processed in such a way that the immune response against the transplant is reduced.

An important feature of graft preparation is that certain structures remain undamaged. The axon is the part of a neuron which conducts electrical impulses. Axons are surrounded by myelin, which contain Schwann cells. Schwann cells improve the electrical conduction of axons. Myelin is surrounded by endoneurium, which is a protective sheath of connective tissue. The endoneurium is surrounded by perineurium and epineurium, of which the latter is the outmost layer of dense connective tissue. For the purpose of surgical nerve repair, it is crucial that those layers retain structural integrity. However, the present disclosure is based on the notion that removal of the extracellular matrix within the epineural space can improve the quality and utility of the remaining structures, i.e., the fascicles and axons disposed therein.

### D. Fusogen Treatment

The present disclosure employs a cell membrane fusogen. The fusogen can be of a chemical nature or a biological nature. Examples include the cell-cell fusogenic glyoproteins (e.g., H-alpha 7) and agents such PEG, chitosan, dextran sulfate, N-nonyl. Bromide, calcium and sodium nitrate.

### 1. Cell-Cell Fusogens

Cell-cell fusogens are glycoproteins that facilitate the fusion of cell-to-cell membranes. Cell-cell fusion is critical for the merging of gamete genomes and the development of organs in multicellular organisms. Cell-cell fusion occurs when both actin cytoskeleton and fusogenic proteins properly rearrange across the cell membrane. This process is led by actin-propelled membrane protrusions.

EFF-AFF are the identifiers for type 1 glycoproteins that make up cell-cell fusogens. They were first identified when EFF-1 mutants were found to “block cell fusion in all epidermal and vulval epithelia” in the roundworm. Caenorhabditis elegans. EFF-AFF is a family of type I membrane glycoproteins that act as cell-cell fusogens, named from ‘anchor cell fusion failure’. Because it was known that EFF-1 mutants successfully fused the anchor cell and (uterine seam) use syncytium to produce a continuous uterine-vulval tube, where these connections failed, AFF-1 mutants were discovered. AFF-1 was deemed necessary for this process in addition to the fusion of heterologous cells in C. elegans. The transmembrane forms of these proteins, like most viral fusogens, possess an N-terminal signal sequence followed by a long extracellular portion, a predicted transmembrane domain, and a short intracellular tail. A striking conservation in the position and number of all 16 cysteines in the extracellular portion of EFF-AFF proteins from different nematode species suggests that these proteins are folded in a similar 3D structure that is essential for their fusogenic activity. C. elegans AFF-1 and EFF-1 proteins are essential for developmental cell-to-cell fusion and can merge insect cells. Thus, FF's comprise an ancient family of cellular fusogens that can promote fusion when expressed on a viral particle. In the medical field, experiments are done to test for the uses of cell-cell fusogens in axonal nerve repairs and to determine their usefulness with other nerve cells. The current method for nerve repair is suturing the cut ends of nerves. This has a long recovery process, with a low functionality rate for the repaired nerves. When considering cell-cell fusogens as a potential answer, researchers divided these fusogens into two groups based on fusion mechanisms: cell aggregation and membrane modification. One fusogen PEG was found to fit in both groups. It was this fusogen that made restoring nerve cells in humans possible. Once operations were within a certain time frame (12 hours for human nerve repair and 24 hours for sciatic rat treatments), patient recovery was almost successful. With this research, there is potential for repairing human nerve grafts. Some potential uses of cell-cell fusogens studied are cancer vaccines and the regeneration of damaged cells. Additionally, any peripheral nerve in the body could be repaired, and transferred tissues could work as soon as the senses return. Finally, any surgery done on nerves could be repaired as well, thus resulting in a quicker recovery.

### 2. Polyethylene Glycol

The inventors have previously reported (Ghergherchchi et al., 2016; Mikesh et al., 2018a,b) a well-defined protocol for the administration of four pharmaceutical agents in solution, one of which contains the plasmalemmal fusogen PEG (Gefter et al., 1977; Lentz and Lee, 1999; Lentz, 2007; Pontecorvo, 1975) as an adjunct to standard neurorrhaphy for primary repair of PNIs. This protocol results in the immediate reconnection (fusion) of the open axonal ends (PEG-fusion) of many axons in closely apposed proximal and distal ends of singly transected nerves (Bittner et al., 2012; Lore et al., 1999; Mikesh et al., 2018a). The same is true for PEG-fused auto- and allografted nerve segments to repair segmental ablation injuries (Bittner et al., 2015; Mikesh et al., 2018b; Riley et al., 2015). In contrast to tissue repair strategies using neurorrhaphy alone, PEG-fusion as an adjunct to neurorrhaphy also repairs many severed cellular (axonal) processes within the PNS nerve tissue (e.g., rodent sciatic nerve).

In conjunction with neurorrhaphy, PEG-fusion of axons in singly cut PNIs or PEG-fusion of axons in autografts/allografts to repair ablation PNIs in a rat sciatic nerve model produces dramatically improved recovery of coordinated, volitional function as measured by the Sciatic Functional Index (SFI: Bittner et al., 2012; de Medinaceli et al., 1982; Wood et al., 2011). The inventors (Mikesh et al., 2018a; 2018b) have also demonstrated that PEG-fusion results in: (a) restoration of axolemmal and axoplasmic continuity and action potential through-conduction across the coaptation site(s) within minutes, (b) prevention of Wallerian degeneration for many axonal segments distal to the coaptation site(s), (c) preservation of distal NMJs indefinitely and prevention of distal target muscle atrophy. (d) recovery of voluntary function (behavior) that occurs rapidly (days to weeks), often to levels seen in unoperated animals, and to substantially improved quality compared to animals repaired with neurorrhaphy alone, and (c) non-rejection of living allogenic allografts in outbred rats in the non-protected environment of a sciatic peripheral nerve with no immunosuppressive treatments. Furthermore, human clinical cases show that the improved speed and quality of sensory recovery of PEG-fused severed digital nerves are very similar to those reported for recovery of function after PEG-fusion of severed sciatic nerves in the rat sciatic injury model (Bamba et al., 2016b).

The table below from Ghergherehchi et al. (2019) shows an exemplary protocol:

PEG-fusion protocol

| Step # | Technique | Purpose(s) |
|---|---|---|
| Preperation | Trim nerve ends | Prepare nerve ends for [symbol] and PEG |
| | | repair fusion |
| 1 - Priming | Irrigation of thew surgical field with #Z,899 | Increase [symbol] volume |
| | Ca² [symbol] for 1-2 min | Open cut [symbol] ends |
| | | Expel intracellular membrane- [symbol] |
| 2 - Protection | Administration of 1% methylene [symbol] (M [symbol] | Prevent [symbol] of in [symbol] cellular [symbol] |
| | antioxidant) in distilled water for 1-2 min [symbol] | than [symbol] with PEG-fusion of [symbol] ends |
| | the [symbol] ends | |
| 3 - Coa [symbol] cut | P [symbol] | Provide mechanical strength to [symbol] to |
| peripheral nerve ends | | prevent PEG- [symbol] |
| | | [symbol] |
| 4 - PEG- [symbol] | Apply 50% w/w [symbol] PEG in distilled | Remove [symbol] cell water to induce [symbol] |
| | water for 1-2 min to the [symbol] | open, [symbol] membranes to [symbol] |
| 5 - Complete | Irrigation of the [symbol] site with [symbol] | [symbol] to [symbol] any [symbol] |
| membrane repair | volume of [symbol] Ca² [symbol] containing saline | [symbol] after PRG-induced annealing of the open [symbol] |
| | | [symbol] ends |

[symbol]

indicates data missing or illegible when filed

### IV. Kits

Also provided are kits comprising reagents suitable for use in storing axons or tissues containing axons. The kits will comprise Normosol-R and polyethylene glycol. The kits may also comprise additional items such as surgical instruments (scalpel, sutures/needles), containers for mixing solutions and/or for treating tissues including nerve grafts. A kit also typically includes a label or packaging insert including a description of the components or instructions for use in vitro, in vivo, or ex vivo, of the components therein.

A kit refers to a physical structure housing one or more components of the kit. Packaging material can maintain the components in a sterile environment and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein. Labels or inserts can also include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter.” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, or an electrical storage media such as RAM and ROM or hybrids of these such as magnetic/optical storage media, FLASH memory, hybrids and memory type cards.

### V. Examples

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Example 1—Materials and Methods

Animals. All experimental procedures were approved by standards set forth by the Institutional Animal Care and Use Committee at the University of Texas at Austin and were conducted in accordance with the guidelines of the National Institutes of Health on the care and use of animals. Sprague-Dawley (SD) rats or Thy1-GFP SD rats (gifted by Dr. Susan Mackinnon, Washington University in St. Louis) of the same sex at ages 3-12 months were housed 2-3/cage and maintained on a 12 h:12 h light:dark cycle with food and water given ad libitum.

Nerve Extraction. Sciatic nerve segments (PNVAs, n=179) were harvested during the rat's active cycle. Female rats weighing 225-300 g or male rats weighing 300-350 g were induced by anesthesia of a 4% isoflurane/oxygen mixture at 1.51/min. Prior to the nerve harvest, all surgical tools were autoclaved. The incision site was first cleanly shaved, followed by sterilization using several wipes of 70% ethanol followed by Betadine. A 4-5 cm incision was made through the biceps femoris to expose the sciatic nerve. Sciatic nerve segments 2.5-3 cm in length were excised and stored in 10 ml of designated storage solutions.

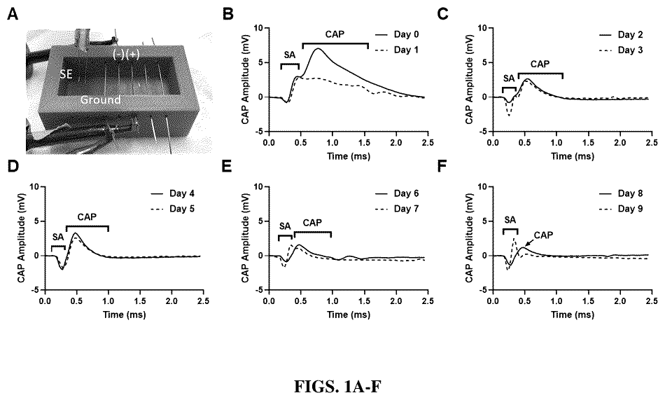

Electrophysiological Testing. CAPs that conducted from one to the other end of each PNVA were recorded ex vivo immediately following harvesting and daily afterward for up to seven days using a PowerLab 4/35 (AD instruments, Sydney, Australia). PNVAs were placed in a customized 3D printed chamber (ABS polymer and Craftbot XL machine) with titanium wires hooked up to a set of stimulating electrodes at one end and a set of recording electrodes at the other end (FIG. 1A). PNVAs were stimulated with incremental increases in voltage from 0-8v using 0.1 ms square wave depolarizations given at 1 Hz with a 0.1 ms delay from a sweep triggering pulse (STP). The minimum threshold and maximum amplitude of CAPs for each PNVA were recorded for each day of graft storage (FIGS. 1B-F). To avoid nerve damage, stimulus amplitudes were limited to 2v maximum on the first day and 8v maximum at longer storage times. The inventors tested for two additional days following the day when no CAP was detected in a PNVA to ensure that the axons in the PNVA were no longer conducting. PNVAs that did not conduct CAPs immediately after harvesting due to either instrumental error or harvesting problems were excluded from the study (n=7).

Storage Solutions. Fresh storage solutions containing 1× final concentration of penicillin-streptomycin (P4333; Sigma-Aldrich, St. Louis, MO, USA) were prepared and replaced daily. PNVAs were stored ex vivo in one of seven different sterile storage solutions: (1) Normosol-R (NR) (0990-7967-09; ICU Medical, San Clemente, CA, USA), (2) diluted Normosol-R (dNR) consisting of NR diluted with 176 ml sterile ddH₂O per liter of NR, (3) diluted Normosol-R with calcium (dNRc) consisting of dNR with 17 mg CaCl₂ per liter of dNR. (4) Lactated Ringers (LR) (2B2323; Baxter Healthcare, Deerfield, IL, USA), (5) Normal Saline (NS) (0990-7983-09; ICU Medical) or made with 900 mg NaCl per liter of ddH₂O and filter-sterilized, (6) diluted Normal Saline (dNS) consisting of NS diluted with 200 ml sterile ddH₂O per liter of NS, and (7) University of Wisconsin Cold Storage Solution (UW) (NC0952695; Fisher Scientific, Hampton, NH, USA). Specifically, 100 ml of each solution contained:
1) NR: 526 mg NaCl, 222 mg C₂H₃NaO₂, 502 mg NaC₆H₁₁O₇, 37 mg KCl, 30 mg MgCl₂
2) dNR: 447 mg NaCl, 189 mg C₂H₃NaO₂, 427 mg NaC₆H₁₁O₇, 31 mg KCl, 26 mg MgCl₂
3) dNRc: same composition as dNR with the addition of 17 mg CaCl₂
4) LR: 600 mg NaCl, 310 mg C₃H₅NaO₃, 30 mg KCl, 20 mg CaCl₂H₄O₂
5) NS: 900 mg NaCl
6) dNS: 750 mg NaCl
7) UW: 5,000 mg Pentafraction, 3583 mg C₁₂H₂₂O_{12, 340} mg KH₂PO₄, 123 mg H14MgO₁₁S. 1783 mg C18H32O₁₆·5H₂₀, 134 mg C₁₀H₁₃N₅O₄, 13.6 mg C₅H₄N₄O, 92 mg C₁₀H₁₇N₃O₆S, 561 mg KOH, 5 mg NaOH (5N)

Solution contained penicillin and streptomycin. All experimental solution protocols are listed below in Table 1. The osmolarity of each was measured many times (>10) on a well-calibrated freezing point osmometer (Model 3300; Advanced Instruments, Norwood, MA, USA). The measured osmolarity of many solutions was often different from their calculated osmolarity given on their commercial labels (Table 1 column 4versus 3). For example, the calculated osmolarity on the label assuming complete dissociation/hydration of NR was 294 mOsm, but the measured osmolarity was 268 mOsm. That is, the total osmotic concentration of many standard solutions is too high to assume complete hydration/no interaction between individual particles in an aqueous solution.

Morphological Analyses. PNVAs of similar length taken from similar regions of the thigh were fixed and embedded similar to previous descriptions (Mikesh et al., 2018; Smith et al., 2020). PNVAs (n=17) were fixed overnight at room temperature in 2% paraformaldehyde/3% glutaraldehyde fixatives (15713, 16220; Electron Microscopy Sciences (EMS), Hatfield, PA, USA) in 0.1 M sodium cacodylate buffer (11653; EMS). Tissues were washed with buffer prior to trimming and post-fixing in 1% osmium tetroxide/1% potassium ferrocyanide (19150; EMS) in 0.1 M sodium cacodylate buffer for 4-5 h. PNVAs were then washed in water, stained in 1% aqueous uranyl acetate (22400-1; EMS) for 2 h, and then washed and held in water. PNVAs were dehydrated through graded ethanol, exchanged to absolute acetone, placed in increasing concentrations of Hard Plus Resin 812 (14115; EMS), and then embedded in fresh resin and polymerized at 60° C. for at least 48 h. Glass knife thick sections (0.5 mm) were stained in toluidine blue, and images were captured by Zeiss Axiovert 200 M microscope using an Axiocam HR3 camera. For each PNVA sample, 3 regions of interest were given blinded to two investigators and manually annotated for a total of at least 150 axons. Five different categories of axons were then reported as mean±s.c.m. percentages of the total number of axons.

Statistical Analyses. All statistical analyses were performed using GraphPad Prism 8.0 software. PNVA survival data were analyzed using the Log-rank test and the Gehan-Breslow-Wilcoxon test. Morphological data were analyzed using two-way ANOVA followed by Tukey's multiple post-hoc test. 95% confidence interval was used. Electrophysiological data were presented as survival curves. PNVA survival times measured by CAPs and percentage of axons counted based on different morphological categories were presented as mean±s.c.m.

### Example 2—Results

Study Design. The current study was designed to determine how long axons in PNVAs remained viable as assessed by their functional ability to conduct action potentials recorded extracellularly (CAPs), as well as their morphological status as assessed by axoplasmic and myelin structures. A pilot study (n=38) indicated no significant differences in the ability of PNVAs to conduct CAPs whether PNVAs were harvested before or after euthanasia with an intracardiac injection of saturated potassium chloride.

The inventors tested four common solutions; several were tested at different temperatures (4° C. vs 25° C.), osmolarities, or calcium concentrations totaling 10 different protocols (Table 1). NR and LR were solutions the inventors were currently using for acute (0-6 h) storage of sciatic PNVAs used for PEG-fusion repair of ablation (segmental-loss) PNIs in previous publications (Riley et al., 2015; Mikesh et al., 2018; Ghergherchchi et al., 2019; Smith et al., 2020a; 2020b). NS is a common solution utilized in clinics, and UW has been used in previous peripheral nerve graft studies as the primary storage solution (Levi et al., 1994; Evans, P. J. et al., 1998).

Electrophysiological Assessments. FIGS. 1A-F shows that as the storage time increased, PNVAs required higher stimulus voltages to elicit a CAP and the amplitude of the conducted CAP decreased. FIGS. 1B-F show an example of recordings from a single preparation of a PNVA in 4° C. dNR. CAPs at storage day 0 and 1 (FIG. 1B) could be induced at low stimulus voltages (0.2-1v). On day 1, the CAP decreased in amplitude but did not shift in its onset time. On days 2-3 (FIG. 1C), CAP amplitude, duration and onset time decreased and required higher voltage (2-6v) to elicit. CAPS at storage days 4-5 were very similar to those of days 2-3, with an even shorter onset time with respect to the SA (FIG. 1D). Starting on day 6 (FIG. 1E), the amplitude of CAP further decreased and required a yet-higher stimulus voltage (6-8v) to elicit. On storage day 8 (FIG. 1F), the CAP was very small and difficult to distinguish from the SA. On storage day 9, no CAP was detectable.

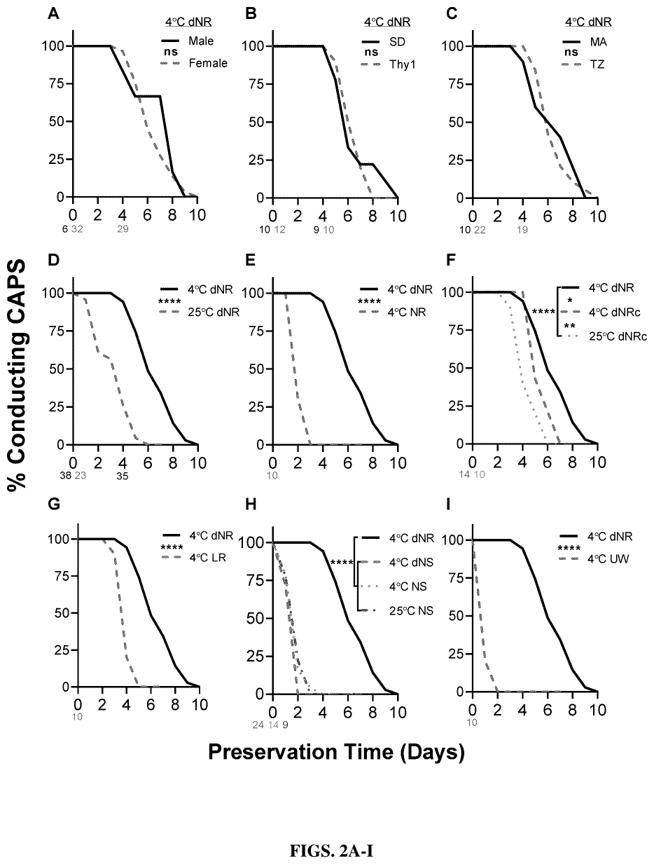

FIGS. 2A-I show survival curves of PNVAs stored in various conditions and solutions assayed by CAP conduction. FIGS. 2A-C shows no significant difference for PNVA survival in 4° C. dNR between (1) female (n=32) and male (n=6) SD rats (FIG. 2A), (2) wild-type SD rats (n=10) and Thy1-GFP SD rats (n=12) (FIG. 2B), or (3) two different operating surgeons (MA (n=10) and TZ (n=22) (FIG. 2C).

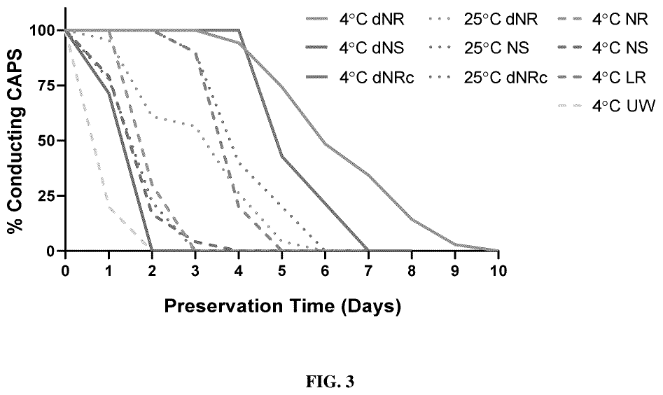

Since no significant differences of PNVA stored in 4° C. dNR were observed for two different sexes, strains, or surgeons, a combined CAP conduction data from FIGS. 2A-C (n=38) was plotted in FIGS. 2D-I. This 4° C. dNR composite curve in FIGS. 2D-I appears to have an S-shaped survival curve over 10 days of storage. PNVAs in 4° C. dNR on average conducted CAPS for 5.7±0.27 days (Table 2). All stored PNVAs conducted CAPs on day 3. On day 4, almost all (94%) of stored PNVAs conducted CAPs. 74% of stored PNVAs conducted CAPs on day 5. Still almost half of all PNVAs (49%) conducted CAPs on day 6. Furthermore, CAPs survived in one PNVA up to 9 days. Because this 4° C dNR composite curve was the best of the inventors' 10 storage solution conditions for maintaining PNVAs (FIG. 3), the inventors used this curve to compare the effects of nerve storage on CAP conduction with all other solutions tested in this study in FIGS. 2D-I as listed below:

FIG. 2D shows that PNVA survival was significantly (p<0.0001, see Table 2) better in 4° C. dNR than in 25° C. dNR (n=23). PNVAs in 25° C. dNR on average conducted for 2.4±0.29 days. While all PNVAs in 4° C. dNR conducted CAPs on day 3, only 57% PNVAs in 25° C. dNR conducted. On day 5, 74% of PNVAs in 4° C. dNR conducted CAPs, while only 4.4% of PNVAs in 25° C. dNR conducted CAPs. All PNVAs in 25° C. dNR stopped conducting CAPs on day 6.

FIG. 2E shows that PNVA survival was significantly (p<0.0001) better in 4° C. hypotonic dNR than in 4° C. isotonic NR (n=10). PNVAs in 4° C. NR on average conducted for 1.3±0.15 days. While all PNVAs in 4° C. dNR conducted CAPs up to day 3, only 30% of PNVAs in 4° C. NR conducted CAPs on day 2. All PNVAs in 4° C. NR stopped conducting on day 3.

FIG. 2F shows that PNVA survival was significantly (p=0.009) impaired in 4° C. dNRc (n=14) and further impaired (p<0.0001) in 25° C. dNRc (n=10) than in 4° C. dNR. PNVAs in 4° C. dNRc on average conducted for 4.6±0.23 days, and those in 25° C. dNRc on average conducted for 3.5±0.31 days. While all PNVAs in 4° C. dNRc conducted CAPs on day 4, more than half of PNVAs stopped conducting on the following day, and by day 6 only 21% of PNVAs continued to conduct CAPs compared to 49% of PNVAs conducting in 4° C. dNR. All PNVAs in 4° C. dNRc stopped conducting on day 7. PNVA survival of CAP conduction is even less when stored in 25° C. dNRc, where only 20% of PNVAs conducted on day 5. All PNVAs in 25° C. dNRc stopped conducting on day 6.

FIG. 2G shows that PNVA survival of CAP conduction was significantly (p<0.0001) better in 4° C. dNR than in 4° C. LR (n=10). PNVAs in 4° C. LR on average conducted for 3.1±0.18 days. On day 3, 90% of PNVAs in 4° C. LR conducted CAPs. However, a sharp drop in CAP conduction was observed on day 4, at which time only 20% of PNVAs were considered viable. All PNVAs in 4° C. LR stopped conducting on day 5.

FIG. 2H shows that PNVA survival of CAP conduction was not optimal in any NS-based storage conditions and was not significantly different among 4° C. NS (n=24), 4° C. dNS (n=14), and 25° C. NS (n=9). CAPs in PNVAs stored in 4° C. NS, 4° C. dNS, and 25° C. NS survived significantly (p<0.0001) shorter than those stored in 4° C. dNR. PNVAs in 4° C. NS, 4° C. dNS, and 25° C. NS on average conducted for 1.0±0.15, 0.7±0.13, and 1.0±0.24 days, respectively. On day 2, while all PNVAs in 4° C. dNR conducted CAPs, all PNVAs in 4° C. dNS stopped conducting, and only 17% of PNVAs in 4° C. NS and 22% of PNVAs in 25° C. NS conducted CAPs. On day 3, only 4.2% of PNVAs in 4° C. NS conducted, while all PNVAs in 25° C. NS stopped conducting. All PNVAs in 4° C. NS stopped conducting on day 4.

FIG. 2I shows that PNVA survival was significantly (p<0.0001) better in 4° C. dNR than in 4° C. UW (n=10). PNVAs in 4° C. UW on average only conducted for 0.2±0.13 days. All PNVAs conducted CAPs immediately following harvest prior to storage in 4° C. UW, but only 20% of PNVAs conducted on day 1. All PNVAs in 4° C. UW stopped conducting on day 2.

FIG. 3 plots on one graph the inventors' electrophysiological survival data for all 10 storage conditions at all storage time points. FIG. 3 shows the following:

At the end of day 1, all PNVAs conducted CAPs in 5 storage conditions: 4° C. dNR, 4° C. NR. 4° C. LR, 4° C. dNRc, and 25° C. dNRc. For other storage conditions, 96% of PNVAs conducted CAPs in 25° C. dNR. 70-80% of PNVAs conducted in 4° C. NS, 4° C. dNS, and 25° C. dNS. Only 20% of PNVAs conducted CAPs in 4° C. UW. At the end of day 4, almost all PNVAs conducted CAPs in 4° C. dNR and 4° C. dNRc (94% and 100%, respectively). Only 20-40% of PNVAs conducted in 4° C. LR, 25° C. dNR, and 25° C. dNRc, and none of the PNVAs stored in the other 5 storage conditions conducted CAPs. At the end of day 7, only PNVAs stored in 4° C. dNR conducted CAPs. At the end of day 10, all PNVAs stopped conducting CAPs in all storage conditions. Overall, 4° C. dNR was the most optimal storage solution among all conditions tested.

Overall, PNVAs conducted CAPs for longer storage times when stored in solutions with lower osmolarity with the exception of dNS at 4° C. For example, PNVAs stored in 4° C. LR conducted CAPs for significantly (p<0.0001) longer times than 4° C. UW (up to day 4 and 1. respectively). Lower temperature improved PNVA storage times in some solution conditions. As discussed above (FIGS. 2D, 2F), PNVAs in either 4° C. dNR or 4° C. dNRc conducted for significantly (p<0.0001, p=0.0077) longer time than their respective counterparts at 25° C. In contrast, PNVAs stored in 4° C. NS and 25° C. NS showed no significant difference in survival times as assayed by CAP conduction. Calcium had variable effects on PNVA storage times at different temperatures. PNVA stored in 4° C. dNR remained viable significantly longer than those in 4° C. dNRc (p=0.009), but the survival curves of PNVAs stored in 25° C. dNR and 25° C. dNRc were not significantly different.

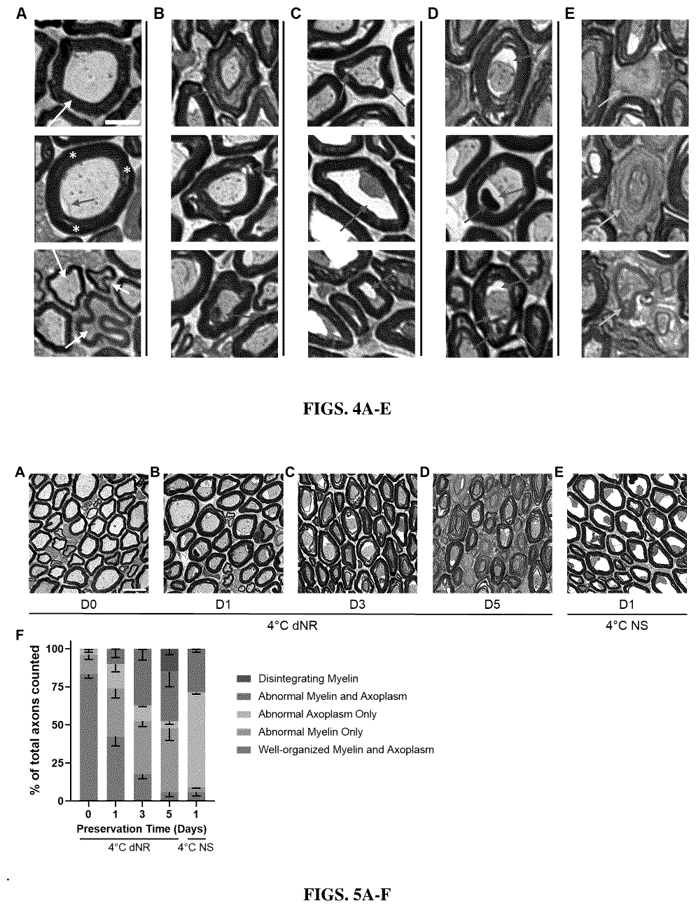

Morphological Assessments. To characterize some of the unique features that represent less severe WD in PNVAs following multiple days of storage, the inventors have developed a five-category classification system to describe axonal morphologies. Those five categories are: (1) Well-organized myelin and axoplasm; (2) Abnormal Myelin; (3) Abnormal axoplasm; (4) Abnormal myelin and axoplasm. 5. Disintegrating myelin. FIGS. 4A-E shows examples of this five-category classification system illustrated in individual panels as follows.

FIG. 4A shows axons that have well-organized myelin and axoplasm that has been termed “intact” in previous publications (Fox et al., 2005; Kerns et al., 2021). The white arrow in the top panel points to a large-diameter myelinated axon having many, non-convoluted myelin layers in which the myelin layers are compact (non-dissociated). The middle panel shows an axon similar to that of the top panel with some minor myelin dissociations (asterisks) and a slight retraction of the axoplasm (blue arrow). These small types of perturbations can even be seen in fresh nerves fixed immediately upon harvest. Therefore, this axon is counted as a well-organized axon in the inventors' classification system. The arrows in the bottom panel point to small-diameter axons having fewer myelin layers that are more convoluted, but well-organized myelin and axoplasm (white arrows).

FIG. 4B shows axons with abnormal myelin sheaths. The red arrow in the top panel points to dissociated myelin wrapped around an axon with well-organized axoplasm. The red arrows in the middle panel point to large spaces (more severe dissociations) within the myelin layers. The red arrow in the bottom panel points to an axon with an extensive myelin intrusion (bleb).

FIG. 4C shows axons with retracted axoplasm surrounded by a well-organized myelin sheath. Some axons have two or more different points of retractions (blue arrow, top panel), or a single, much more pronounced retraction (blue arrows, middle and bottom panels).

FIG. 4D shows axons that contain abnormalities present in both the myelin and axoplasm, to varying degrees of severity. The top panel shows an axon with severe myelin dissociation (red arrow) and axoplasmic retraction (blue arrows). The middle panel shows an axon with myelin intrusion (red arrow) and axoplasm retraction (blue arrow). The bottom panel shows an axon with myelin dissociation (right red arrow), myelin intrusion (left red arrow), and axoplasm retraction (blue arrows).

FIG. 4E shows axons with disintegrating myelin that is very poorly stained with toluidine blue (orange arrows). Since the visualization of myelin sheaths is achieved by osmium tetroxide binding to the double bonds in lipids, the faint staining of these axons suggests a severe degradation of myelin sheath. Some axons completely lose all heavily stained myelin (top panel), while some retain remnants of their disassociating myelin sheath (middle and bottom panel).

Comparing Morphological Assessments in Different Solutions. To assess the morphological correlates of PNVAs in 4° C. dNR, the inventors sampled PNVAs on 0, 1, 3, and 5 d of storage (FIGS. 5A-E). The inventors compared morphological data for 4° C. dNR (the solution with the best CAP survival curve in FIG. 3) with those for PNVAs stored 4° C. NS (one of the storage solutions with the worst CAP survival curve) on day 1, since that is the last day when CAPs in most PNVAs remained viable in NS. Table 3 shows percentages of axons for each morphological category within each group; Table 4 shows statistical comparisons for each morphological category between groups.

Most axons in PNVAs in 4° C. dNR fixed immediately following tissue harvest on storage day 0 (FIG. 5F) had well-organized myelin and axoplasm (83±2.7%), a few had abnormal myelin (13±3.3%) or abnormal axoplasm (3.2±1.8%), and almost none had abnormal myelin and axoplasm (0.6±0.6%). None had disintegrating myelin (Table 3).

As the storage time in 4° C. dNR increased, PNVAs exhibited increased signs of morphological degradation (FIG. 5F). Two-way ANOVA interaction was statistically significant (p<0.0001), indicating storage duration affects axonal composition of PNVAs. The percentage of well-organized axons decreased significantly in PNVAs stored in 4° C. dNR (p<0.0001; see Table 4), starting from 83±2.7% (day 0) to 42±6.2% (day 1) to 18±3.1% (day 3) to 6.0±3.1% (day 5) as reported in Table 3. This decrease correlated with a decrease in CAP amplitude from days 0-5 (FIGS. 1A-F). In addition, as summarized in Table 3, the percentages of axons with abnormal myelin significantly increased between day 0 and 5 (13±3.3% to 42±8.0%; p=0.0005). Similarly, the percentages of axons with both abnormal myelin and axoplasm significantly increased between day 0 and 5 (0.6±0.6% to 33±9.9%; p<0.0001). The percentage of axons with abnormal axoplasm was not statistically different between day 0 and 5 (3.2±1.8% to 4.4±1.9%). Finally, the percentages of axons with disintegrating myelin were negligible between day 0 to 3 (all <0.4±0.4%) until day 5 at which time an increase (15±4.0%) was noted that was not significant (p=0.15).

As summarized in Table 3, PNVAs stored in 4° C. NS on day 1 contained only 5.9±2.6% axons with well-organized myelin and axoplasm (FIG. 5F). A few had abnormal myelin (3.1±0.5%) or disintegrating myelin (0.9±0.3%). The majority of the axons had abnormal axoplasm (62±1.0%) or both abnormal myelin and axoplasm (28±1.5%).

Statistical differences were observed between PNVAs stored in 4° C. dNR and 4° C. NS on day 1 (Table 4). While 42±6.2% of axons remained well-organized in 4° C. dNR, only 5.9±2.6% axons in 4° C. NS were well-organized (p<0.0001). 4° C. dNR contained significantly more axons with abnormal myelin (31±6.1% vs. 3.1±0.5%; p=0.0007), while 4° C. NS contained significantly more axons with abnormal axoplasm (62±1.0% vs. 16±5.1%; p<0.0001). These differences were maintained when comparing PNVAs stored in 4° C. NS to those in 4° C. dNR until day 3 and day 5 (p=0.0003 or less; see Table 4). Although not statistically significant (p=0.06), a trend of more axons with both abnormal myelin and axoplasm was also observed in PNVAs stored in 4° C. NS compared to those in 4° C. dNR on day 1. Interestingly, PNVAs stored in 4° C. NS on day 1 exhibit similar small percentages of well-organized axons as do those in 4° C. dNR on day 5 (5.9±2.6% vs. 6.0±3.1%).

TABLE 1
Storage Solutions

| | | Calculated | Measured | # of |
|---|---|---|---|---|
| | | Osmolarity | Osmolarity | nerve |
| Solution | ° C. | (mOsmol) | (mOsmol) | samples |
| NR | 4° | 294-295 | 268 | 10 |
| dNR | 4° | 250 | 250-255 | 38 |
| | 25 | 250 | 250-255 | 23 |
| dNRc | 4° | 250 | 250-255 | 14 |
| | 25 | 250 | 250-255 | 10 |
| LR | 4° | 273 | 264 | 10 |
| NS | 4° | 300-308 | 290-295 | 24 |
| | 25 | 300-308 | 290-295 | 9 |
| dNS | 4° | 250 | 250-255 | 14 |
| UW | 4° | 320 | 318 | 10 |

Solution acronym, temperature, calculated osmolarity, measured osmolarity, and number of nerves harvested for that protocol.
Range of measured osmolarity indicates differences across different batches.

TABLE 2
Summary Statistics and Statistical Comparisons of PNVA Survival Times

| Comparison (Survival time in days) | | | | | Log-rank test | Gehan-Breslow-Wilcoxon test |
|---|---|---|---|---|---|---|
| 4° C. dNR (5.7 ± 0.27) | | vs | 25° C. dNR (2.4 ± 0.29) | | p < 0.0001 | p < 0.0001 |
| | | | 4° C. NR (1.3 ± 0.15) | | p < 0.0001 | p < 0.0001 |
| | | | 4° C. dNRc (4.6 ± 0.23) | | p = 0.0087 | p = 0.0200 |
| | | | 25° | C. dNRc | p < 0.0001 | p < 0.0001 |
| | | | 4° C.LR (3.1 ± 0.18) | | p < 0.0001 | p < 0.0001 |
| | | | 4° C. NS (1.0 ± 0.15) | | p < 0.0001 | p < 0.0001 |
| | | | 4° C. dNS (0.7 ± 0.13) | | p < 0.0001 | p < 0.0001 |
| | | | 25° C. NS (1.0 ± 0.24) | | p < 0.0001 | p < 0.0001 |
| | | | 4° C. UW (0.2 ± 0.13) | | p < 0.0001 | p < 0.0001 |
| 25° | C. dNR | vs | 4° | C. NR | p = 0.0060 | p = 0.0272 |
| | | | 4° | C. dNRc | p < 0.0001 | p < 0.0001 |
| | | | 25° | C. dNRc | ns | ns |
| | | | 4° | C. LR | ns | ns |
| | | | 4° | C. NS | p < 0.0001 | p = 0.0003 |
| | | | 4° | C. dNS | p = 0.0001 | p = 0.0001 |
| | | | 25° | C. NS | p = 0.0018 | p = 0.0058 |
| | | | 4° | C. UW | p < 0.0001 | p < 0.0001 |
| 4° | C. NR | vs | 4° | C. dNRc | p < 0.0001 | p < 0.0001 |
| | | | 25° | C. dNRc | p < 0.0001 | p < 0.0001 |
| | | | 4° | C. LR | p < 0.0001 | p < 0.0001 |
| | | | 4° | C. NS | ns | ns |
| | | | 4° | C. dNS | p = 0.0101 | p = 0.0109 |
| | | | 25° | C. NS | ns | ns |
| | | | 4° | C. UW | p = 0.0004 | p = 0.0003 |
| 4° | C. dNRc | vs | 25° | C. dNRc | p = 0.0077 | p = 0.0051 |
| | | | 4° | C. LR | p < 0.0001 | p < 0.0001 |
| | | | 4° | C. NS | p < 0.0001 | p < 0.0001 |
| | | | 4° | C. dNS | p < 0.0001 | p < 0.0001 |
| | | | 25° | C. NS | p < 0.0001 | p < 0.0001 |
| | | | 4° | C. UW | p < 0.0001 | p < 0.0001 |
| 25° | C. dNRc | vs | 4° | C. LR | ns | ns |
| | | | 4° | C. NS | p < 0.0001 | p < 0.0001 |
| | | | 4° | C. dNS | p < 0.0001 | p < 0.0001 |
| | | | 25° | C. NS | p < 0.0001 | p < 0.0001 |
| | | | 4° | C. UW | p < 0.0001 | p < 0.0001 |
| 4° | C. LR | vs | 4° | C. NS | p < 0.0001 | p < 0.0001 |
| | | | 4° | C. dNS | p < 0.0001 | p < 0.0001 |
| | | | 25° | C. NS | p < 0.0001 | p < 0.0001 |
| | | | 4° | C. UW | p < 0.0001 | p < 0.0001 |
| 4° | C. NS | vs | 4° | C. dNS | ns | ns |
| | | | 25° | C. NS | ns | ns |
| | | | 4° | C. UW | p = 0.0013 | p = 0.0011 |
| 4° | C. dNS | vs | 25° | C. NS | ns | ns |
| | | | 4° | C. UW | p = 0.0150 | p = 0.0150 |
| 25° | C. NS | vs | 4° | C. UW | p = 0.0106 | p = 0.0101 |

Statistical pair-wise comparisons summarizing PNVA CAP survival plotted in FIG. 3 for the Log-rank test and Gehan-Breslow-Wilcoxon test.
ns, not significant.
PNVA CAP survival times are expressed as mean ± s.e.m.
ns = not significant

TABLE 3
Summary Statistics of Morphological Analyses

| | | | | Abnormal | |
|---|---|---|---|---|---|
| | Well- | Abnormal | Abnormal | Myelin ± | Disintegrating |
| | organized | Myelin | Axoplasm | axoplasm | Myelin |
| Day 0 4° C. dNR (n = 3) | 83 ± 2.7 | 13 ± 3.3 | 3.2 ± 1.8 | 0.60 ± 0.60 | 0.0 ± 0.0 |
| Day 1 4° C. dNR (n = 4) | 42 ± 6.2 | 31 ± 6.1 | 16 ± 5.1 | 9.9 ± 5.6 | 0.30 ± 0.17 |
| Day 3 4° C. dNR (n = 3) | 18 ± 3.1 | 35 ± 3.7 | 10 ± 0.76 | 37 ± 7.1 | 0.37 ± 0.37 |
| Day 5 4° C. dNR (n = 4) | 6.0 ± 3.1 | 42 ± 8.0 | 4.4 ± 1.9 | 33 ± 9.9 | 15 ± 4.0 |
| Day 1 4° C. NS (n = 3) | 5.9 ± 2.6 | 3.1 ± 0.50 | 62 ± 0.98 | 28 ± 1.5 | 0.88 ± 0.29 |

Summary statistics of PNVA morphology.
Percentages of axons in each morphological category in each group are expressed as mean ± s.e.m.

TABLE 4
Statistical Comparisons of Morphological Analyses

| | | | | | | Abnormal | |
|---|---|---|---|---|---|---|---|
| | | | Well- | Abnormal | Abnormal | Myelin ± | Disintegrating |
| Comparison | | | organized | Myelin | Axoplasm | axoplasm | Myelin |
| Day 0 | vs | Day 1 | p < 0.0001 | ns (p = 0.0551) | ns | ns | ns |
| 4° C. | | 4° C. | | | | | |
| dNR | | dNR | | | | | |
| | | Day 3 | p < 0.0001 | p = 0.0250 | ns | p < 0.0001 | ns |
| | | 4° C. | | | | | |
| | | dNR | | | | | |
| | | Day 5 | p < 0.0001 | p = 0.0005 | ns | p < 0.0001 | ns |
| | | 4° C. | | | | | |
| | | dNR | | | | | |
| | | Day 1 | p < 0.0001 | ns | p < 0.0001 | p = 0.0025 | ns |
| | | 4° C. NS | | | | | |
| Day 1 | vs | Day 3 | p = 0.0038 | ns | ns | p = 0.0012 | ns |
| 4° C. | | 4° C. | | | | | |
| dNR | | dNR | | | | | |
| | | Day 5 | p < 0.0001 | ns | ns | p = 0.0041 | ns |
| | | 4° C. | | | | | |
| | | dNR | | | | | |
| | | Day 1 | p < 0.0001 | p = 0.0007 | p < 0.0001 | ns | ns |
| | | 4° C. NS | | | | | |
| Day 3 | vs | Day 5 | ns | ns | ns | ns | ns |
| 4° C. | | 4° C. | | | | | |
| dNR | | dNR | | | | | |
| | | Day 1 | ns | p = 0.0003 | p < 0.0001 | ns | ns |
| | | 4° C. NS | | | | | |
| Day 5 | vs | Day 1 | ns | p < 0.0001 | p < 0.0001 | ns | ns |
| 4° C. | | 4° C. NS | | | | | |
| dNR | | | | | | | |

Statistical pair-wise comparisons summarizing PNVA morphology.
Two-way ANOVA followed by Tukey's multiple comparison's test.
Adjusted p values are listed.
ns = not significant.

Example 3—Discussion

Summary of electrophysiological and morphological results. This study used an electrophysiological (CAP recordings) criterion to assess the viability of PNVAs stored ex vivo. First, CAP recordings of PNVAs were not significantly affected by the identity of the operating surgeon, the euthanasia procedure, and the strain or sex of the rat, even though sciatic PNVAs have somewhat larger diameters in males than females. Second, PNVA storage times were significantly improved by four conditions: (1) solution composition, (2) reduced tonicity, (3) lower temperature, and (4) low calcium concentration. Condition (1) and possibly (2) had stronger effects than (3-4). That is, as one example of the effect of solution composition, PNVAs stored in 4° C. LR conducted CAPs for a significantly longer time (up to 4 days) than those stored in 4° C. UW (up to 1 day). As an example of the effect of reduced tonicity, PNVAs stored in 4° C. dNR conducted CAPs for a significantly longer time (up to 9 days) than those stored in 4° C. NR (up to 2 days). As an example of the effects of lower temperature, PNVAs stored in 4° C. dNR conducted CAPs for a significantly longer time (up to 9 days) than those stored in 25° C. dNR (up to 5 days). As an example of the effects of low calcium concentration, PNVAs stored in 4° C. dNR conducted CAPs for a significantly longer time (up to 9 days) than those stored in 4° C. dNRc (up to 6 days).

The inventors' detailed morphometric assessment method reports the distinct morphological categories for PNVAs in 4° C. dNR, including well-organized axons, axons with abnormal myelin dissociation or intrusion, axons with retracted axoplasm, axons with both abnormal myelin and axoplasm, and axons with disintegrating myelin. As storage time increased, the percentage of well-organized axons decreased significantly, but a small population of these axons still existed on day 5. The percentage of axons with abnormal myelin increased between day 0 and 1 but remained unchanged beyond day 1. Similarly, the percentage of axons with abnormal myelin and axoplasm increased between days 0 and 3 but remained unchanged beyond day 3. The percentage of axons with abnormal axoplasm did not vary significantly at any time point tested and was always low. Axons with disintegrated myelin sheath, indicative of tissue degradation, appeared on day 5. In contrast, PNVAs in 4° C. NS exhibited a small population of well-organized axons on day 1, while the majority of the axonal population exhibited either abnormal axoplasm or abnormal myelin and axoplasm.

Most importantly, the inventors have demonstrated, for the first time, preservation solutions and protocols that maintain axon viability in PNVAs for several days after harvest. The inventors have just begun to examine how different storage solutions might affect axonal viability in PNVAs. The inventors do not know how different cellular/biochemical mechanisms or pathways might be affected by differences in the four conditions (solution composition, measured osmotic strength, temperature, and presence of calcium) examined in this study. Other solutions (e.g., HTK, DMEM, Collin's solution) reported as best for the storage of other types of tissue should also be investigated because they control additional variables such as the pH, sugars, amino acids, and electrolytes (Collins and Wicomb, 1992; Lassner et al., 1995; Spiegel et al., 1999; Jing et al., 2018) not examined in this study.

Storage solutions optimal for non-neuronal organs and tissues versus PNVAs. Storage solutions optimized for the storage of non-neuronal organs and tissues are not necessarily optimal for PNVA storage. For example, NS solution is often optimally used to store non-neuronal tissue grafts, including bone and vein grafts (Steiner and Ramp, 1988; Davies and Hagen, 1994). UW solution, created to prevent ischemic stress, is commonly used as an optimal solution to store non-neuronal organs, such as kidneys, livers, and pancreatic tissues (Belzer and Southard, 1988; Voigt and DeLario, 2013). Most unexpectedly in this study, 4° C. NS or UW were decidedly suboptimal for PNVA storage as assessed by CAPs conduction, i.e., CAPs conducted for only 1 or 2 days. UW had previously been tested for nerve storage (Evans et al., 1998; Fox et al., 2005; Matsumoto et al., 2005; Ikeguchi et al., 2007), and intact myelin structures were observed after 7 weeks of cold storage. However, axonal viability was never tested. Among all the unmodified solutions tested in this study at 4° C. (UW, NS, NR, and LR), LR maintained PNVA viability in PNVA for the longest storage times.

Storage solutions tonicity as a variable. Lower osmolarity of the storage solution often led to longer PNVA storage times with the exception of dNS at 4° C. For example, PNVAs stored in 4° C. dNR (250-255 mOsm) continued to conduct CAPs up to 9 days but only for 2 days in 4° C. NR (268 mOsm). The inventors' morphological data also suggested higher tonicity may lead to retraction of axoplasm and axolemma from the myelin sheath. In 4° C. dNR (250-255 mOsm), 37% of axons exhibited retracted axoplasm after 5 days of storage, whereas 90% of axons exhibited retracted axoplasm just after 1 day of storage in 4° C. NS (290-295 mOsm). This may explain why UW performed the worst in PNVA storage at 4° C. UW contains lactobionic acid and raffinose, which were specifically added to create hypertonicity and cause cell desiccation prior to cryopreservation (Belzer and Southard, 1988; Chen et al., 2019). This principle of design may be detrimental to axonal viability in PNVA storage.

Storage temperature as a variable. Lower temperature often led to better PNVA storage times. For example, when assayed for CAP conduction, 4° C. dNR and 4° C. dNRc outperformed 25° C. dNR and 25° C. dNRc, respectively. The inventors' result is consistent with previous reports of tissue storage (including nerve allografts) at different cold temperatures to reduce cell metabolism and activities and tissue degradation (Belzer and Southard, 1988; Levi et al., 1994; Evans et al., 1999; Fox et al., 2005; Ostrozka-Cieslik et al., 2018). However, temperature was not necessarily the primary determinant variable for axonal viability since 4° C. NS and 25° C. NS had similar PNVA storage times based on CAP conduction. Additionally, 4° C. UW, NS, dNS, and NR all had poorer PNVA storage times for viable CAP conduction than dNR at 25° C.

Calcium concentration as a variable. PNVA storage time assayed by CAP conduction was longer in calcium-free dNR at 4° C. than calcium-containing dNRc at 4° C. This result was consistent with (Schlaepfer, 1974) in which calcium-containing solutions induced granular disintegration of axoplasmic microtubules and neurofilaments in stored rat nerve segments. However, calcium, like temperature, was not the dominant factor for axonal viability when comparing different solutions of different compositions, osmolarities, and temperatures when assessed by CAP conduction. As one example, 4° C. UW had poorer PNVA storage times than 4° C. calcium containing LR. As another example, 25° C. dNR had poorer PNVA storage times than 4° C. dNRc.

Previous axonal morphometric studies on Wallerian degeneration. The inventors know of no previous publications describing axonal morphometrics for nerves maintained ex vivo, perhaps because nerve grafts are often regarded simply as mechanical conduits to guide axonal regeneration. Previous morphometrics studies have characterized WD in vivo. For example, (Kerns et al., 2021) described three (ABC) categories of myelinated axons during WD: spared, partial/moderate degenerated, and advanced degenerated. However, that classification system did not separate populations of axons with obvious morphological differences in myelin and axoplasm and did not capture all the characteristics displayed in stored nerves demonstrated in this study.

PNVAs versus conventional conduits used in repairs of segmental-loss PNIs. Axons in nerve grafts have been expected to undergo WD, and the collapsed basal lamina tubes have been expected to guide axonal regeneration by outgrowth from the host proximal nerve with the help of supporting cells (ref). Hence, previous nerve storage studies focused on maintaining the viability of the supporting cells rather than axons and the integrity of the graft structure. For example, (Lassner et al., 1995) compared cold preservation of rat nerve grafts in various solutions to assess Schwann cell and fibroblast growth ex vivo, axon counts and myelination following transplantation. These repair strategies using various conduit materials always led to slow and poor behavioral recovery.

As an alternate and distinct approach, the inventors propose to use PNVAs that are PEG-fused to repair segmental-loss PNIs. In PEG-fused PNVAs, the open cut ends of axons at both ends of the donor PNVA are joined to/fused with open cut ends of host axons that are still viable at the proximal and distal ends of the gap where the PNVA has been inserted (Riley et al., 2015). When compared to conduits, autografts, or other current growth guides, PEG-fused PNVAs have the following advantages: 1) PNVAs can be selected to be predominantly motor, sensory, or mixed sensory/motor, depending on the nature of the lesioned nerve. 2) PNVAs can be size- or shape-matched to the lesioned nerve. 3) PNVAs do not produce additional host morbidity as do autografts. 4) PNVAs exhibit complex biological features of intact peripheral nerves. 5) PEG-fused PNVAs need not be tissue matched or immune suppressed to avoid immune rejection and restore many voluntary behaviors within weeks (Bittner et al., 2016b; Bittner et al., 2018; Mikesh et al., 2018b; Smith et al., 2020; Roballo et al., 2022).

Conclusions. Maintaining axonal viability in PNVAs is important because viable donor axons in PNVAs can be PEG-fused with viable proximal and distal ends of host axons to repair segmental-loss peripheral nerve injuries that produce excellent recovery of sensory/motor functions and voluntary behaviors within weeks. Such PEG-fused PNVAs, unlike other donor transplants, are immune-tolerated without tissue matching or immune suppression. An inability to store PNVAs for more than a day would be a limiting factor to expand the potential of PEG-fusion technology to repair segmental-loss PNIs. Therefore, longer PNVA storage times could enable the establishment of PNVA tissue banks and the use of stored, PEG-fused PNVAs to repair segmental-loss PNIs. Furthermore, optimal storage solutions and conditions for PNVAs might be relevant for the storage of other donor tissue grafts that contain nerves, such as limb transplants or even heart transplants.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A nerve storage solution comprising calcium-free solution comprising sodium chloride, sodium acetate anhydrous, sodium gluconate, potassium chloride, and magnesium chloride and exhibiting no more than about 250-255 mOsm per liter (measured osmosality).

2. The nerve storage solution of claim 1, wherein sodium chloride is present at about 526 mg per 100 ml.

3. The nerve storage solution of claim 1, wherein sodium acetate is present at about 222 mg per 100 ml.

4. The nerve storage solution of claim 1, wherein sodium gluconate is present at about 502 mg per 100 ml.

5. The nerve storage solution of claim 1, wherein potassium chloride is present at about 37 mg per 100 ml.

6. The nerve storage solution of claim 1, wherein magnesium chloride hexahydrate is present at about 30 mg per 100 ml.

7. canceled

8. The nerve storage solution according to claim 1, wherein said nerve storage solution is further defined as:
(a) comprising hydrochloric acid and/or sodium hydroxide;
(b) having a pH range 6.5 to 7.6;
(c) having electrolytes per 1000 mL (not including pH adjustment) of sodium about 140 mEq, potassium about 5 mEq, magnesium about 3 mEq, chloride about 98 mEq, acetate about 27 mEq, and gluconate about 23 mEq; and
(d) (i) comprising no bacteriostat, antimicrobial agent or added buffer (except for pH adjustment) or (ii) comprising a bacteriostat or antimicrobial agent, with or without added buffer, such as one or more of penicillin, streptomycin, amphotericin B, kanamycin, gentamicin sulfate, tylosin, chloramphenicol, ampicillin, neomycin, benzylpenicillin, hygromycin B, carbenicillin, erythromycin, cefotaxime, paromomycin and fosmidomycin, or combinations thereof, including penicillin+streptomycin.

9. The nerve storage solution of claim 1, wherein the nerve storage solution is hypotonic Normosol-R (dNR).

10. The nerve storage solution according to claim 1, further comprising an axon or a tissue comprising an axon.

11. A method of repairing an injury to a nerve site in a subject comprising:
(a) providing a nerve graft comprising an axon;
(b) treating said nerve graft with a solution according to claim 1;
(c) placing the nerve graft into the injured nerve site.

12. The method of claim 11, wherein the injured nerve site is in a peripheral nerve.

13. The method of claim 11 wherein the injured nerve site is in an organ, such as heart, lung, kidney, liver, colon, intestine, esophagus, tongue, or stomach.

14. The method according to claim 11, wherein the injured nerve site is in an arm, a leg, a finger or a toe.

15. The method according to claim 11, wherein the nerve graft is derived from a sensory nerve, motor nerve, or a mixed nerve.

16. The method according to claim 11, wherein the nerve graft is an allograft, such as a vascularized composite allograft, isograft or an autograft.

17. The method according to claim 11, wherein the subject is a human subject.

18. The method according to claim 11, wherein the subject is a non-human mammalian subject.

19. The method according to claim 11, wherein the injury is an acute injury or a chronic injury.

20. The method of claim 11, wherein the nerve graft is treated with polyethylene glycol.

21. A method of treating or storing an axon or tissue containing an axon comprising contacting said axon or tissue containing an axon with a nerve storage solution comprising calcium-free solution comprising sodium chloride, sodium acetate anhydrous, sodium gluconate, potassium chloride, and magnesium chloride and exhibiting no more than about 250-255 mOsm per liter (measured osmosality).

22.-34. canceled

35. An axon or tissue comprising an axon treated with a nerve storage solution according to claim 1.
